(19) United States
(12) Patent Application Publication
(10) Pub. No.: US 2022/0293224 A1
(43) Pub. Date: Sep. 15, 2022

(54) MODULAR SYNTHON-BASED SCREENING APPROACH FOR USE IN DRUG DISCOVERY FOR DISEASES

(71) Applicant: UNIVERSITY OF SOUTHERN CALIFORNIA, Los Angeles, CA (US)

(72) Inventors: Vsevolod Katritch, Irvine, CA (US); Arman Sadybekov, San Diego, CA (US)

(21) Appl. No.: 17/691,958

(22) Filed: Mar. 10, 2022

Related U.S. Application Data
(60) Provisional application No. 63/159,888, filed on Mar. 11, 2021.

(51) Int. Cl. G16C 20/50; G16C 20/64

(52) U.S. Cl. CPC G16C 20/50; G16C 20/64

(57) ABSTRACT

This disclosure provides for modular synthon-based screening for rapid drug discovery. Such screening includes initially docking a pre-built set of fragment-like compounds representing library reaction scaffolds and corresponding synthons. Best selected scaffold and synthon combinations from the initial docking are used to enumerate a further library, which is screened again to produce fully enumerated compounds. Such an iterative approach focuses on a subset of synthons at each screening, thereby reducing the combinatorial chemical space for docking and facilitating more rapid drug discovery.

### CROSS-REFERENCE TO RELATED APPLICATIONS

This application is based upon and claims priority to U.S. provisional patent application 63/159,888 entitled “MODULAR SYNTHON-BASED SCREENING APPROACH FOR POTENTIAL USE IN DRUG DISCOVERY FOR DISEASES” and filed on Mar. 11, 2021, the entire content of which is incorporated herein by reference.

### STATEMENT AS TO FEDERALLY SPONSORED RESEARCH

This invention was made with government support under grants R01DA041435 and R01DA045020 by the National Institute on Drug Abuse and grant R01MH112205 by the National Institute of Mental Health. The government has certain rights in this invention.

### BACKGROUND

### 1. Field

This disclosure relates generally to drug discovery, and more specifically, to modular synthon-based screening for drug discovery.

### 2. Description of the Related Art

Standard libraries for high-throughput (HTS) and virtual ligand screening (VLS) have been historically limited to about 1-10 million available compounds, which is a minute fraction of the enormous chemical space of an estimated 10²⁰ to 10⁶⁰ drug-like compounds. This limitation of standard HTS and VLS slows the pace of drug discovery as, for example, smaller screens usually yield initial hits with a modest affinity (˜(micromolar), poor selectivity and ADMET profiles, and require elaborate multistep optimization to gain lead- and drug-like candidate properties. Structure-based virtual ligand screening is emerging as a key paradigm for early drug discovery owing to the availability of high-resolution target structures and ultra-large libraries of virtual compounds. With increasing library sizes, the computational time and cost of docking-based VLS itself become the next bottleneck in screening, even with the massively parallel cloud computing capacities. For example, screening of 10 Billion compounds at a standard docking rate of 10 second/compound would take >3000 years on a single CPU core, or cost >$800,000 at a rate of 3¢ per CPU core hour on a computing cloud, making it largely impractical. Thus, there remains a need to dramatically reduce the computational burden of VLS, without compromising the accuracy of docking or losing the best hit compounds to remove this bottleneck and assure accessibility of such giga-scale screening to researchers.

### SUMMARY

A method for efficiently screening of large libraries of compounds is provided. The method is useful to identify the best compounds that dock to receptors for potential use in drug discovery for diseases. The method includes generating a list of proxy compounds including reaction scaffolds and enumerated with corresponding synthons only in a first R position while a second R position is capped with a minimal synthon cap to become a capped R position. The method also includes docking the proxy compounds to the target receptor structure by docking (such as energy-based or empirical, or other docking) of a flexible ligand to predict binding scores and ligand-receptor interaction information and to select a first set of best-scoring proxy compounds. The method contemplates iteratively enumerating the first set of best-scoring proxy compounds so that at least one capped R position is replaced with a full range of corresponding synthons to produce fully enumerated compounds. The method further involves performing docking for the fully enumerated compounds in at least two R positions to select a first set of best docking compounds.

In various embodiments, the minimal synthon cap is methyl or phenyl. The first R position may be only R1 or only R2 for two-component compounds. The large libraries of compounds may include Enamine REadily AvailabLe for synthesis (REAL) compound libraries, REAL Space compound libraries, or any other libraries that can be defined as a limited set of Markush scaffolds with two or more R-groups (synthons). The second R position may be capped with a minimal synthon cap because the reaction scaffolds are often highly polar or charged. The method may also include filtering or screening the first best set of proxy compounds for diversity. The filtering or screening may include an additional compound diversity rule that a single reaction cannot contribute more than 20% of the selection.

In various embodiments, docking the compounds to the target receptor structure further includes selecting of compounds with higher chances for successful enumeration, as defined by distances to specific atoms of a pocket. The iteratively enumerating may include a single iteration for two-component reactions with only two R groups. The iteratively enumerating may include a plurality of iterations for three-component reactions with three R groups. The iteratively enumerating may include repeatedly enumerating a plurality of iterations when the compounds are 4- and 5-component compounds until the compounds are fully enumerated with library synthons. The performing the docking for the fully enumerated compounds may further include filtering for physical-chemical properties, drug-likeness, novelty, and chemical diversity to select a final set of best docking compounds for synthesis and testing that is a subset of the first set of best docking compounds. In various embodiments, the receptors are a cannabinoid CB₁ receptor and a cannabinoid CB₂ receptor. In various embodiments, the receptors include one or more ROCK1 kinase receptor. The receptors may have receptor structures represented by 3D coordinates of the receptor atoms.

A computer-readable medium (CRM) is provided. The CRM may store instructions that when executed by a processor cause the processor to perform a method for using the processor to efficiently screen of large libraries of compounds to identify the best compounds that dock to receptors, for potential use in drug discovery for diseases. The method may include generating a list of proxy compounds having reaction scaffolds and enumerated with corresponding synthons only in a first R position while a second R position is capped with a minimal synthon cap to become a capped R position. The method may include docking the proxy compounds to the target receptor structure by docking (such as energy-based or empirical, or other docking) of a flexible ligand to predict binding scores and ligand-receptor interaction information and to select a first set of best-scoring proxy compounds. The method may include iteratively enumerating the first set of best-scoring proxy compounds so that at least one capped R position is replaced with a full range of corresponding synthons to produce fully enumerated compounds. The method may include performing docking for the fully enumerated compounds in at least two R positions to select a first set of best docking compounds.

In various instances, the minimal synthon cap is methyl or phenyl. The first R position may be only R1 or only R2 for two-component compounds. The method may also include filtering or screening the first best set of proxy compounds for diversity. In various embodiments, the receptors are a cannabinoid CB₁ receptor and a cannabinoid CB₂ receptor. In various embodiments, the receptors include one or more ROCK1 kinase receptor.

A method may be provided. The method may be for efficiently screening of large libraries of compounds to identify the best compounds that dock to at least one of a cannabinoid CB₁ receptor and a cannabinoid CB₂ receptor. The method may include generating a list of proxy compounds having reaction scaffolds and enumerated with corresponding synthons in a first R position and a synthon cap in second R position comprising a capped R position. The method may include docking the proxy compounds to at least one of a cannabinoid CB₁ receptor and a cannabinoid CB₂ receptor by docking (such as energy-based or empirical, or other docking) of a flexible ligand to select a first set of best-scoring proxy compounds. The method may include iteratively enumerating the first set of best-scoring proxy compounds so that at least one capped R position is replaced with a full range of corresponding synthons to produce fully enumerated compounds. The method may include performing docking of the fully enumerated compounds in at least two R positions to select compounds that dock to at least one of a cannabinoid CB₁ receptor and a cannabinoid CB₂ receptor.

### BRIEF DESCRIPTION OF THE DRAWINGS

Other systems, methods, features, and advantages of the present invention will be or will become apparent to one of ordinary skill in the art upon examination of the following figures and detailed description. Additional figures are provided in the accompanying Appendix and described therein.

FIG. 1A illustrates a method for efficiently screening of large libraries of compounds to identify the best compounds that dock to receptors, in accordance with various embodiments;

FIG. 1B illustrates a system for efficiently screening of large libraries of compounds to identify the best compounds that dock to receptors, in accordance with various embodiments;

FIG. 1C-1F provide diagrams illustrating aspects of the method of FIG. 1A, in accordance with various embodiments;

FIGS. 2A-D illustrate rules for structure-guided selection of docked fragments amenable for further enumeration, in accordance with various embodiments;

FIG. 3A illustrates a comparison graph comparing screening performance of V-SYNTHES with standard VLS over the range of docking score thresholds for a 2-component scenario, in accordance with various embodiments;

FIG. 3B illustrates a comparison graph comparing screening performance of V-SYNTHES with standard VLS over the range of docking score thresholds for a 3-component scenario, in accordance with various embodiments;

FIG. 3C shows a graph illustrating enrichment in V-SYNTHES vs. standard VLS at different score thresholds for a 2-component scenario, in accordance with various embodiments;

FIG. 3D shows a graph illustrating enrichment in V-SYNTHES vs. standard VLS at different score thresholds for a 3-component scenario, in accordance with various embodiments;

FIGS. 4A-B depict graphs showing functional characterization of the best V-SYNTHES hits in Tango antagonist assay at a CB₁ receptor, in accordance with various embodiments;

FIGS. 4C-D depict graphs showing the best V-SYNTHES hits at a CB₂ receptor, in accordance with various embodiments;

FIG. 5 shows experimentally identified hit compounds and associated chemical structures, in accordance with various embodiments; and

FIGS. 6A-F show binding poses for various top CB₂ hits identified by V-SYNTHES, in accordance with various embodiments.

### DETAILED DESCRIPTION

Structure-based virtual ligand screening is emerging as a key paradigm for early drug discovery owing to the availability of high-resolution target structures and ultra-large libraries of virtual compounds. However, to keep pace with the explosive growth of virtual chemical libraries, new approaches to compound screening are needed. This disclosure presents a highly scalable synthon-based approach, V-SYNTHES, which performs hierarchical structure-based screening of readily available for synthesis (REAL) combinatorial libraries. This approach includes identifying best synthon-scaffold combinations as seeds suitable for further growth, then iteratively elaborating these seeds to select complete molecules with the best docking scores. This hierarchical combinatorial approach allows rapid detection of the best-scoring compounds in the chemical space of more than 10 billion compounds while performing docking of only a small fraction (˜2 million) of the library. In an example computational assessment for cannabinoid CB₂ receptors screening, the V-SYNTHES final iteration set, as provided further herein, is ˜250 fold enriched in high-scoring hits for the 2-component chemical space and ˜460 fold enriched for the 3-component space. Moreover, chemical synthesis and experimental testing of cannabinoid antagonists predicted by V-SYNTHES demonstrate a 33% hit rate, with a majority of the hits in sub-micromolar range and the best compounds having Kᵢ=50 nM at C₁ and 90 nM at CB2. These results exceed those obtained by a standard virtual screening of the Enamine REAL library diversity subset, which required ˜100 times more computational resources. The approach is scalable for the rapid growth of combinatorial libraries and adaptable for any docking algorithms.

Standard libraries for high-throughput (HTS) and virtual ligand screening (VLS) have been historically limited to about 1-10 million available compounds, which is a minute fraction of the enormous chemical space of an estimated 10²⁰ to 10⁶⁰ drug-like compounds. This limitation of standard HTS and VLS slows the pace of drug discovery as, for example, smaller screens usually yield initial hits with a modest affinity (˜micromolar), poor selectivity and ADMET profiles, and require elaborate multistep optimization to gain lead- and drug-like candidate properties. Recently, ultra-large libraries of more than 100 million readily accessible (REAL) compounds have been developed and employed in docking-based VLS, yielding high quality hits and showing great utility in streamlining lead discovery. The REAL libraries have grown to billions of compounds and are accessible via the ZINC database. The REAL libraries take advantage of modular parallel synthesis with a large set of optimized reactions and building blocks. This makes the synthesis of potential hits fast (less than 4-6 weeks), reliable (>80% success rate) and affordable.

The modular nature of REAL libraries is supportive of their further rapid growth, for example, “Enamine REAL Space” has already expanded beyond 10 billion drug-like compounds. With increasing library sizes, the computational time and cost of docking-based VLS itself become the next bottleneck in screening, even with the massively parallel cloud computing capacities. For example, screening of 10 billion compounds at a standard docking rate of 10 second/compound would take >3000 years on a single CPU core, or cost >$800,000 at the standard rate of 30¢ per CPU core hour on a computing cloud, making it largely impractical. The ability to dramatically reduce the computational burden of VLS, without compromising the accuracy of docking or losing the best hit compounds would remove this bottleneck and assure accessibility of such giga-scale screening to industry and academic researchers. Most importantly, it would accommodate the further rapid growth of the VLS libraries and thus help improve coverage of the chemical space and the overall quality and diversity of the VLS hits for drug discovery. One of the suggested approaches to tackle libraries of this size is stepwise filtering of the whole enumerated library using docking algorithms of increasing accuracy. VirtualFlow, for example, recently allowed screening of ˜1.4 Billion Enamine REAL compounds and yielded submicromolar KEAP1 inhibitors. This screen, however, still required vast computational resources (160,000 CPU on GCP), which scale at least linearly with the size of library. Moreover, the use of simplified fast docking algorithms at the initial steps may eliminate the best potential hits from further consideration.

This disclosure presents a so-called virtual synthon hierarchical enumeration screening (V-SYNTHES) approach that takes full advantage of the modular building block organization of the Enamine REAL Space, does not need full enumeration of the library, and requires at least 100 times less computational resources than standard VLS without compromising docking accuracy at any steps. Moreover, the algorithm scales linearly with the number of building blocks (or “synthons”), or as square or cubic root of the fully enumerated library size (O(N^{1/2}) and O(N^{1/3}) for 2-component and 3-component reactions respectively). Such performance of V-SYNTHES relies on the initial docking of a pre-built set of the fragment-like compounds dubbed the “Minimally Enumerated Library” (MEL) representing all of the library reaction scaffolds and corresponding synthons. The best selected scaffold/synthon combinations from the initial MEL screening are used then to enumerate next-generation focused libraries, which are screened again to produce fully elaborated hits. Such an iterative approach focuses only on a small fraction (<1%) of the best synthons at each enumeration step, thus drastically reducing the combinatorial chemical space for docking.

In an example implementation, the approach is applied to CB₁ and CB₂ cannabinoid receptors, which are class A G-protein coupled receptors (GPCRs), comprising key components of the endocannabinoid system. Modulation of cannabinoid signaling is a key target in drug discovery for inflammatory, neurodegenerative diseases, and cancer. Prospective application of V-SYNTHES using a CB₂ structural template shows that this approach can speed up docking-based detection of the best-scoring hits in a 10 billion library more than 5000 fold, as compared to full VLS. Moreover, experimental validation revealed that the success rate in the discovery of CB hits (Kᵢ<10 μM) by V-SYNTHES exceeded the success rate as compared to a standard VLS screen of the REAL library diversity subset of 115 million cmpds (33 vs 15% respectively), though V-SYNTHES required 100 times less computational resources for docking. The new approach provides a practical alternative for fast screening of modular virtual libraries of more than 10 Billion compounds, helping to identify leads suitable for fast optimization in the same combinatorial space.

In an example implementation, the V-SYNTHES approach has been implemented based on the REAL Space virtual library that comprised more than 11 billion readily accessible compounds based on optimized one-pot parallel synthesis (Enamine), involving 121 reaction protocols and 75,000 unique reagents. In various embodiments, continuing growth of the REAL Space virtual library provides a library comprising more than 21 billion readily accessible compounds. The reaction protocols include single and multistep procedures involving two (102 reaction protocols), three (17 reaction protocols), and four (2 reaction protocols) starting reagents. In this disclosure, examples include use of 2-component and 3-component reactions yielding ˜500 million and ˜10.5 billion compounds respectively. The disclosed V-SYNTHES approach can be easily expanded to 4- and more component reactions. Each reaction/scaffold in the library is presented in the form of a Markush scheme with two or more R-groups, or “synthons.”

High diversity of the REAL space is achieved through utilizing diverse sets of starting reagents. Average numbers of starting reagents per protocol are the following: for 2-reagent reactions, 3,344 (reagent 1) and 2,068 (reagent 2); for 3-reagent reactions, 939 (reagent 1), 1,308 (reagent 2), and 1,389 (reagent 3); for 4-reagent reactions, 43, 57, 423 and 9 (reagents 4).Different numbers may also be contemplated.

The modular design of the library based on well-established and optimized reactions and an automated one-pot parallel synthesis approach allows fast synthesis (less than 4-6 weeks), with a high success rate (>80%) and guaranteed high purity (>90%).

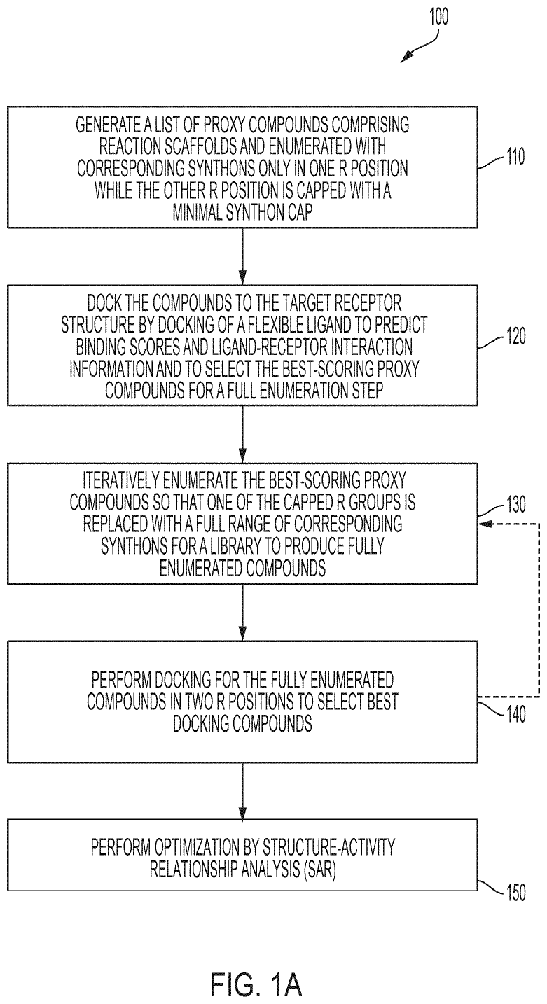

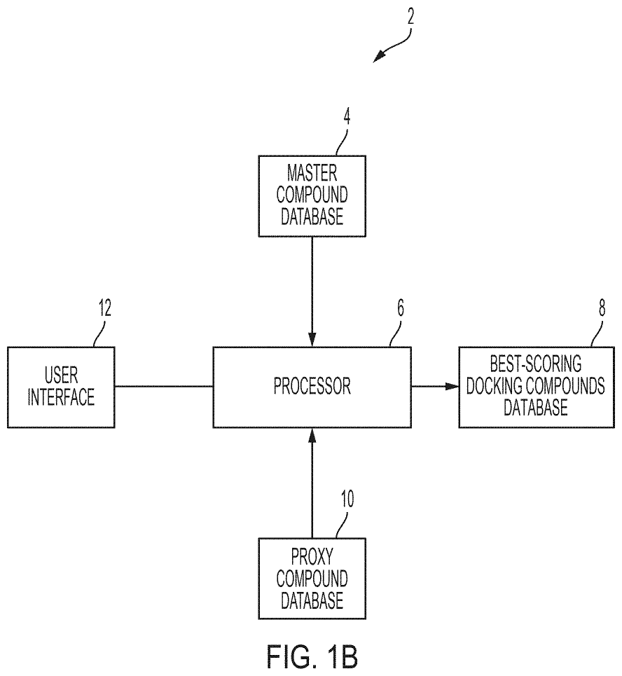

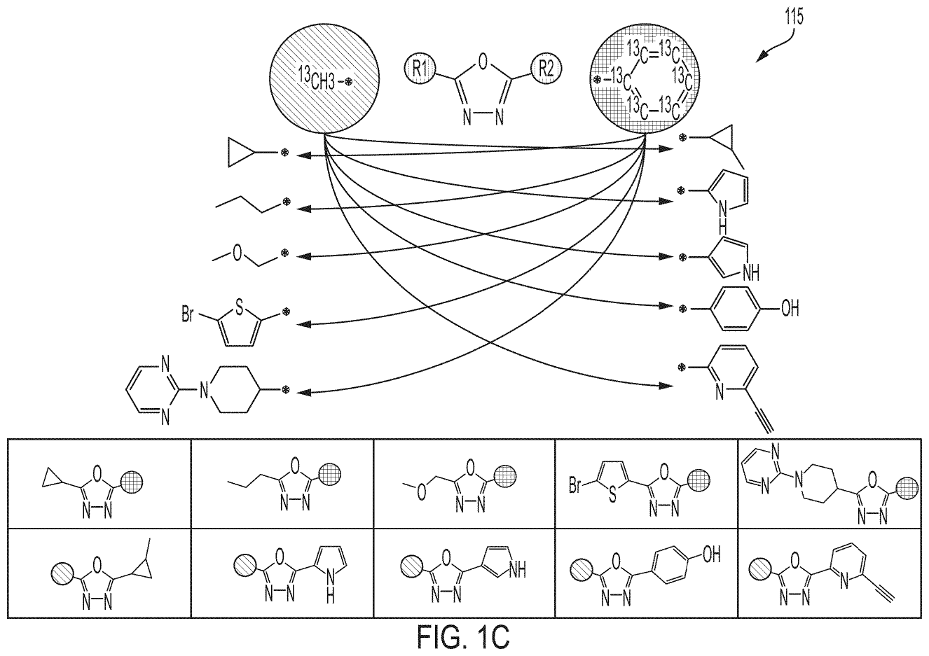

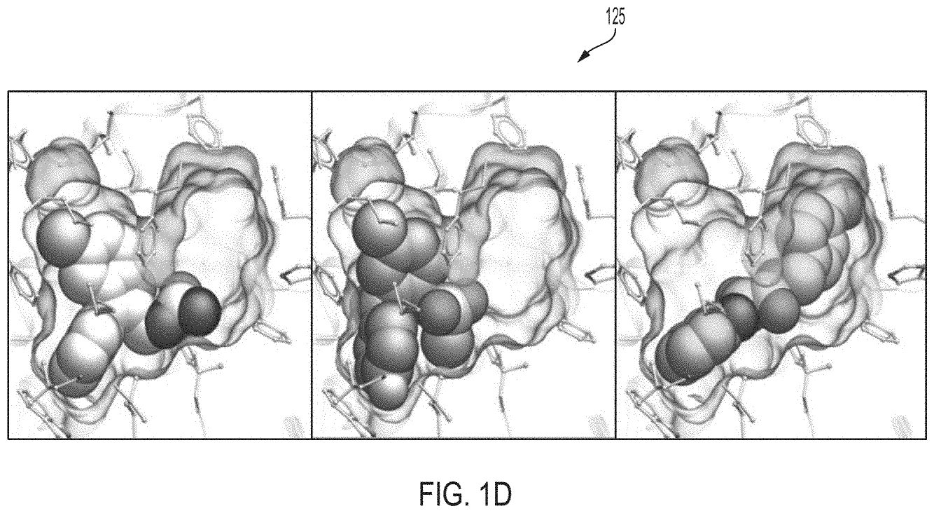

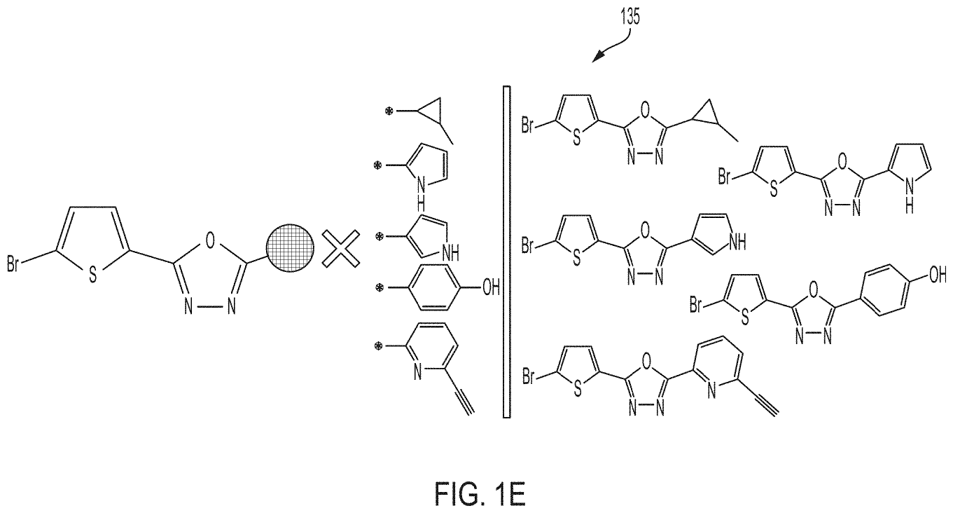

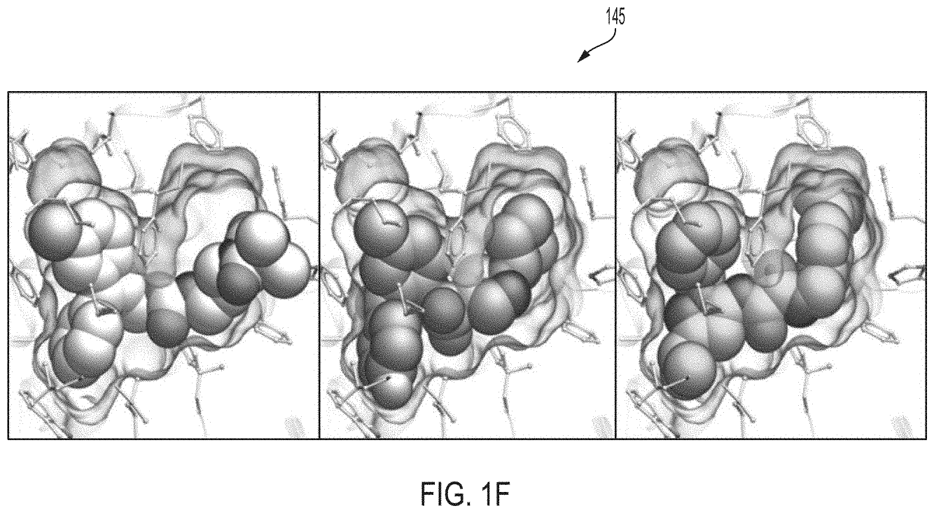

With reference to FIG. 1A, a method 100 is provided for efficiently screening of large libraries of compounds to identify the best compounds that dock to receptors, for potential use in drug discovery for diseases. The method may include iterative steps of library preparation, enumeration, docking, and hit selection.

For example, the method may include generating a list of proxy compounds comprising reaction scaffolds and enumerated with corresponding synthons only in one R position while the other R position is capped with a minimal synthon cap such as methyl or phenyl (block 110). This may be a preparatory step of generating a library of fragment-like compounds representing all possible scaffold-synthon combinations for all reactions in the whole Enamine REAL Space, which we will refer to as a “Minimal Enumeration Library” (MEL). With reference to FIGS. 1A, 1C, an illustration of this aspect is provided in a diagram 115. The MEL compounds are built from the reaction scaffolds, enumerated with the corresponding synthons at one of its R-positions, while the other R-position(s) are being capped by special “minimal” groups selected for each scaffold. The minimal groups, usually one or few atoms (i.e. methyl or phenyl), are needed to “cap” reactive groups of the scaffold that are often highly polar or charged and may distort docking results. Since only one of the R groups is fully enumerated, and others are just systematically “capped”, the MEL library size is approximately equal to the number of synthons in the REAL Space, i.e. only about 600K compounds. This MEL preparation step is performed once for the REAL Space library and does not depend on the target receptor.

The method may also include a docking aspect. For instance, the method may include docking the compounds to the target receptor structure by docking (such as energy-based or empirical, or other docking) of a flexible ligand to predict binding scores and ligand-receptor interaction information and to select the best-scoring proxy compounds for the full enumeration step (block 120). More specifically, the compounds of MEL are docked to the target receptor by docking (such as energy-based or empirical, or other docking) of the flexible ligand. The results of docking, including predicted binding scores and ligand-receptor interaction information, typically a few thousand top-scoring compounds, are then used to select the most promising fragments for the next enumeration. With reference to FIGS. 1A, 1D, an illustration of this aspect is provided in a diagram 125. The selection may also be filtered for diversity, including a rule that a single reaction cannot contribute more than X % of the selection. In various embodiments, X % is 20 percent.

The method may include the iterative enumeration and docking of the best MEL compounds selected in block 120. More specifically, the method may include iteratively enumerating the best-scoring proxy compounds so that one of the capped R groups is replaced with a full range of corresponding synthons for a library to produce fully enumerated compounds (block 130).

On each iteration, the compounds are enumerated so that one of the capped R groups is replaced by a full range of corresponding synthons from the library. For example, for two-component reactions with only two R groups, a single iteration completes the molecule, representing a full compound from the REAL Space. For three- and more component reactions, two and more iterations are performed, replacing the minimal caps with real R group synthons one by one. Thus, each “hit” MEL compound selected in the previous step is iteratively “grown”, resulting in fully enumerated compounds from the REAL Space. With reference to FIGS. 1A, 1E, an illustration of this aspect is provided in a diagram 135.

The method may include performing docking for the fully enumerated compounds in two R positions to select the best docking compounds (block 140). More specifically, the method may include performing on the final enumerated subset of the library. The several thousands of top-ranked VLS hits undergo postprocessing filtering for PAINS, physical-chemical properties, drug-likeness, novelty, and chemical diversity to select a final limited set (typically 50-100) compounds for synthesis and experimental testing. With reference to FIGS. 1A, 1F, an illustration of this aspect is provided in a diagram 145.

The premise of this approach is to enrich the MEL library in connection with aspects performed in block 120 and illustrated in diagram 125 (FIG. 1D), then each subsequent iteration library, with Scaffold-Synthon combinations that have high binding scores in the pocket and are suitable for further enumeration. Because of the modular combinatorial nature of the REAL Space library, narrowing down the most promising scaffolds-synthon combinations dramatically reduces the chemical search space. In a test case, an example used selection parameters that required docking of just 2 million molecules, but still representing the whole 11 billion chemical space. Importantly, the number of docked molecules in V-SYNTHES grows approximately linearly with the number N of synthons in REAL Space library, while the library itself can grow as fast as N^{C} power growth, where C is the number of reaction components (currently 2 or 3).

In various embodiments, the method may include performing optimization by structure-activity relationship analysis (SAR) (block 150). This analysis further optimizes hits identified by the method because the combinatorial nature of the vast library of compounds ensures thousands of close analogues for structure-activity relationship analysis (SAR), and SAR-by-catalogue searching within the library. Further embodiments may omit block 150. Aspects of block 150 are provided in greater detail in later paragraphs.

With reference to FIGS. 1A and also 1B, the method 100 may be performed by a system 2 for efficiently screening of large libraries of compounds to identify the best compounds that dock to receptors, for potential use in drug discovery for diseases. Different components of the system may perform the various iterative steps of library preparation, enumeration, docking, and hit selection. Notably, throughout this disclosure, the terms “library” and “database” may be used interchangeably.

For example, a system may include a master compound database 4. The master compound database may include a database of all potential compounds to dock to receptors. The master compound database 4 may comprise a local computer memory storage device, or a remote database such as a cloud resource.

The system may include a proxy compound database 10. The proxy compound database may include proxy compounds comprising reaction scaffolds and enumerated with corresponding synthons only in one R position while the other R position is capped with a minimal synthon cap such as methyl or phenyl. This proxy compound database may include a library of fragment-like compounds representing all possible scaffold-synthon combinations for all reactions. The proxy compound database may also be termed a minimal enumeration library (MEL), as discussed herein. The proxy compound database 10 may comprise a local computer memory storage device, or a remote database such as a cloud resource.

The system may include a processor 6. The processor may comprise a computer processor, or a cloud computing resource, or a collection of parallel processors working in concert, or any other electronic processor as desired. The processor may load data and/or operating instructions from one or more computer-readable medium. The processor 6 may receive data from the master compound database 4 and the proxy compound database 10. A user may direct operation of the processor via a user interface 12 such as a browser session, a local control session at a human-machine interface, or a remote connection such as across a network. The processor 6 may perform the method 100 disclosed herein and output best docking compounds to a best docking compounds database 8.

The system may include the best docking compounds database 8, as mentioned. The best docking compounds database may receive the best docking compounds identified by the method 100. The best docking compounds database may include a local computer memory storage device, or a remote database such as a cloud resource.

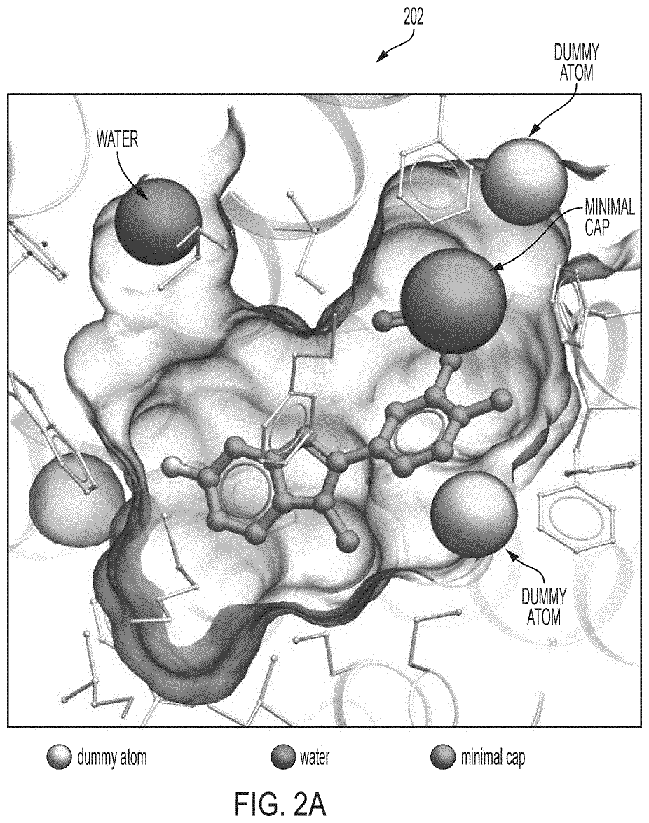

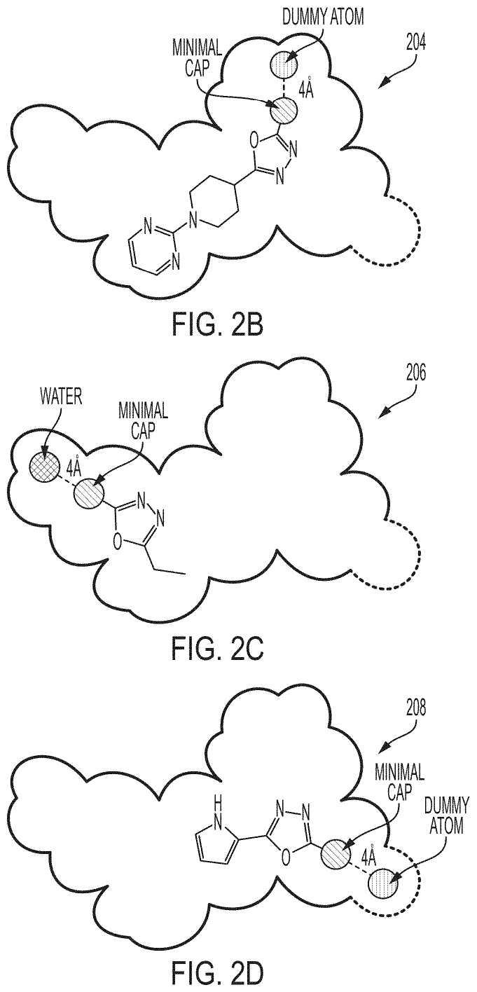

Turning now to FIGS. 2A-D, structure-guided selection of docked fragments amenable for further enumeration may be implemented. Selection of synthons, based solely on binding scores, can already bring substantial library enrichment, with an estimated up to 40 enrichment of high-scoring compounds in the final iteration step than in the full library. At the same time, the performance of the iterative approach can be further improved by taking into account docking poses of the compounds, and specifically, locations of the minimal capping R-group. Thus, docking of the fragments into a binding pocket can result in two conceptually different outcomes. The first, “productive” outcome, is when the minimal capping group of the docked MEL ligand is positioned in the pocket in such a way that it can be replaced by real, bulkier synthons from the library upon the next step of enumeration. This requires the cap to be pointing toward the unoccupied part of the pocket and not being blocked by the pocket residues. A second, “non-productive” outcome is when the minimal cap at one of the R-positions is directly pointing towards the residues at the dead-end sub-pocket, where it does not have space to grow. Another non-productive situation is when the capping R-group is pointing outside of the pocket, where useful contacts are much less likely. To select productive hits, an automated procedure is implemented that checks a distance from the cap atoms to selected dummy atoms or water molecules at the dead-end subpockets. FIGS. 2A-D illustrate corresponding rules 202, 204, 206, 208 in implementation for the CB₂ receptor. The docked MEL compounds for which their cap atoms approached the “dead-end” residues closer than 4A were excluded from further consideration even if they have high-ranked binding scores. FIG. 2A illustrates a 3D illustration 202 of a MEL compound with a non-productive pose. FIGS. 2B-C illustrate other possible non-productive cases including dead-end sub-pockets 204, 206. FIG. 2D illustrates a non-productive case 208 corresponding to an out-of-pocket case.

The approach herein may be implemented for CB₂ receptor virtual screening. The V-SYNTHES approach was applied to screen 11 billion compounds at cannabinoid receptors using recently solved representative CB₂R structure in complex with an antagonist (PDB:5ZTY) as a template. Screening was performed for 2-component and 3-component reactions of the Enamine REAL Space separately, representing ˜500M and ˜10.5B virtual compounds. Note that the V-SYNTHES approach involved docking of just 1M and 0.5M compounds respectively for these libraries in the last enumeration step, reducing the computational cost of screening more than 5000-fold.

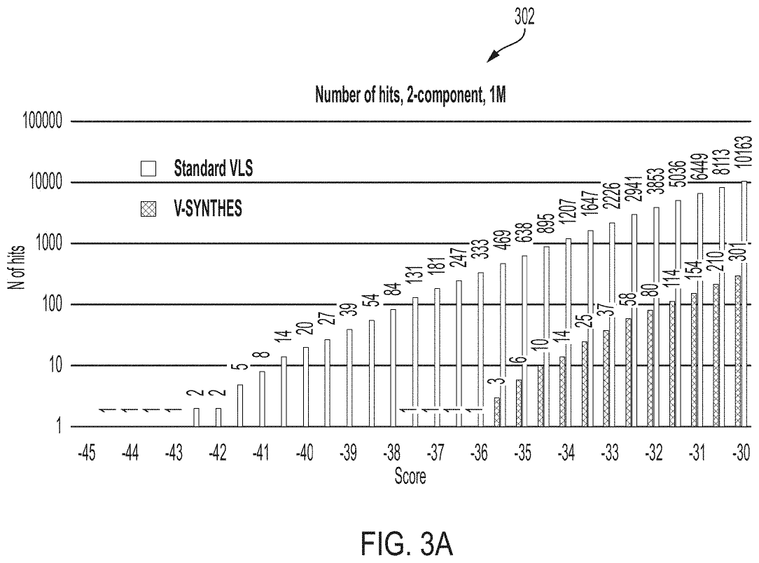

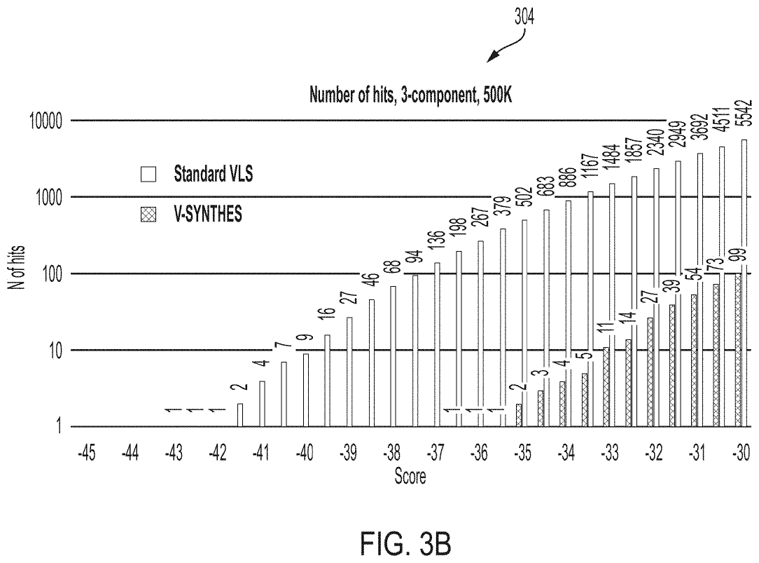

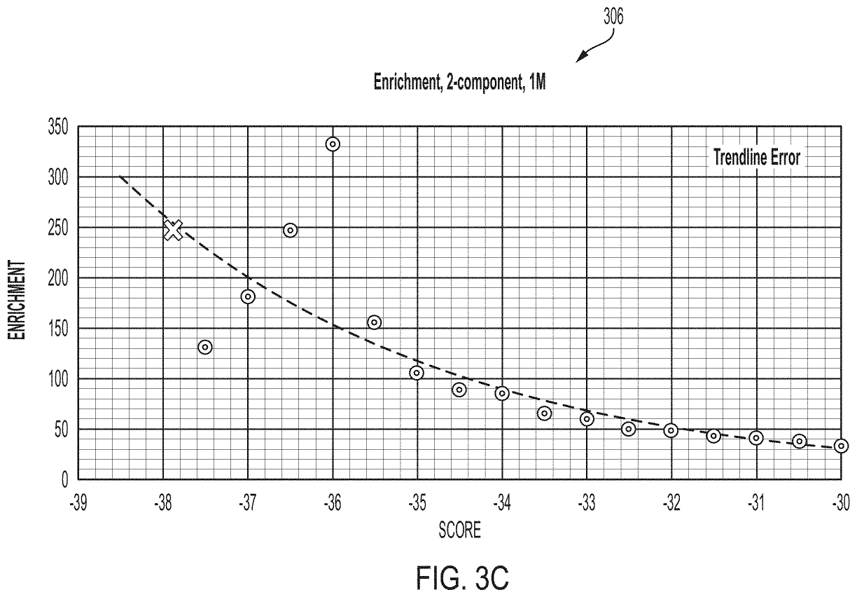

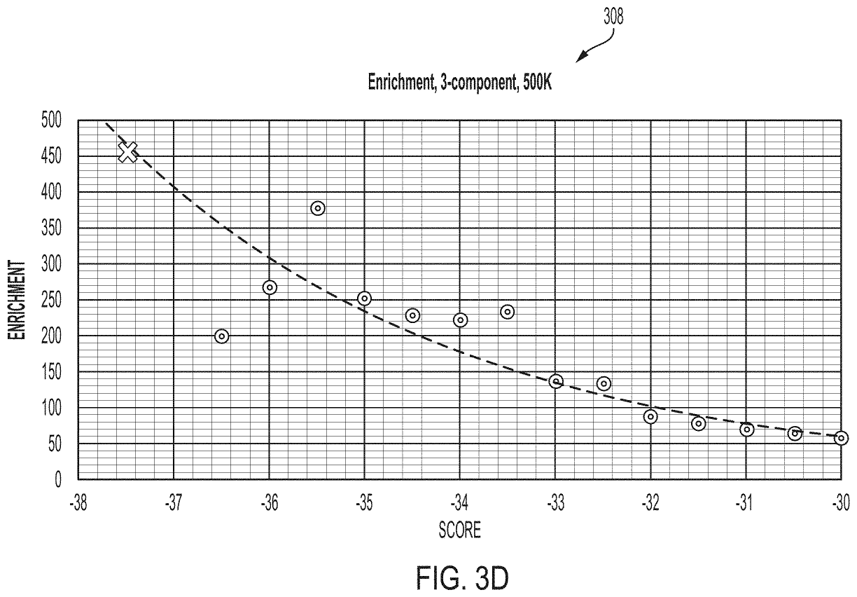

To computationally benchmark performance of the V-SYNTHES approach disclosed herein versus a standard VLS procedure, an example implementation also generated randomized screening libraries of 1M and 0.5M compounds from the same 2-component and 3-component REAL chemical spaces and assessed them in standard VLS using the same receptor model and same docking parameters. FIG. 3A illustrates a comparison graph 302 comparing screening performance of V-SYNTHES with standard VLS over the range of docking score thresholds for a 2-component scenario, and FIG. 3B illustrates a comparison graph 304 comparing screening performance of V-SYNTHES with standard VLS over the range of docking score thresholds for a 3-component scenario. In both instances, results show that V-SYNTHES detected many more high-scoring compounds with much better scores than standard VLS that involved docking of the same number of compounds. Thus, the best 2-component compound identified by V-SYNTHES scored 7 kJ/mol better than the very best hit from the standard VLS; the difference was 6.5 kJ/mol for 3-component compounds. Moreover, in the 2-component REAL space V-SYNTHES identified 84 compounds with binding scores that were better than the very best compound from standard VLS; this number was 136 for the 3-component space.

To systematically characterize the enrichment for high-scoring compounds in the final step of V-SYNTHES versus a random subset of the whole library, this disclosure introduces a an enrichment factor, calculated as a ratio of “number of candidate hits” at a given score threshold for the two libraries, as shown FIGS. 3C, 3D. FIG. 3C shows a graph 306 illustrating enrichment in V-SYNTHES vs. standard VLS at different score thresholds, with the x-mark showing thresholds that yields 100 hits in 2 component cases. FIG. 3D shows a graph 308 illustrating enrichment in V-SYNTHES vs. standard VLS at different score thresholds, with the x-mark showing thresholds that yields 100 hits in 3 component cases.

Note that at −30 kJ/mol binding score threshold, V-SYNTHES already yields ˜40-50-fold higher number of “potential hits” from 2-component (>10,000 hits) and 3-component space (>5,000 hits), compared to standard VLS. This enrichment further increases for more restrictive thresholds, reflecting the V-SYNTHES focus on the iterative selection of the very best-scoring compounds. One relevant threshold for measuring enrichment factors selects the top 100 compounds (referred to herein as EF₁₀₀), where 100 is a typical number of compounds from VLS campaigns to select for synthesis and experimental testing. For a 2-component reaction, this enrichment factor was estimated as EF₁₀₀=250. This is approaching a theoretical limit of “ideal enrichment” ˜500, which would be achievable if all possible hits from the full chemical space of 500M compounds were present in the 1M compound final enumerated library. For the 3-component reactions, the EF₁₀₀=460 is even higher and sufficient for high practical utility, though further from the theoretical limit of 20,000.

The enrichment factor evaluation does not take into account computational efforts for the initial docking of MEL compounds (and intermediate library for 3-component). However, these initial steps add only limited computational costs to V-SYNTHES screens ˜20% for 2-component and 35% for 3-component), because smaller fragment-like compounds in MEL library dock much faster on average than the larger and more flexible compounds. Considering the full computational cost at all the iterative steps, the speedup of V-SYNTHES as compared to standard screening for identification of the 100 top candidate hits at the same score threshold thus can be evaluated as ˜200 fold for 2-component and 300-fold for 3-component compounds in the current benchmark.

The approach herein may be implemented for selection and synthesis of candidate hits for CB receptors. To select the best V-SYNTHES hits for chemical synthesis and in-vitro testing at CB receptors, an example implementation employs a standard post-processing procedure to the top-ranking 5000 candidate hits, which included (i) filtering out compounds with potential PAINS properties and low drug-likeness, (ii) filtering out compounds with high similarity to known CB₁/CB₂ ligands in ChEMBL, (iii) redocking initial hits at a higher docking effort, (iv) clustering and selection of a limited number of best compounds from each cluster to maintain higher diversity of the final set. The final selected set included 80 compounds, of which 60 were synthesized with >90% purity and delivered by Enamine in less than 5 weeks.

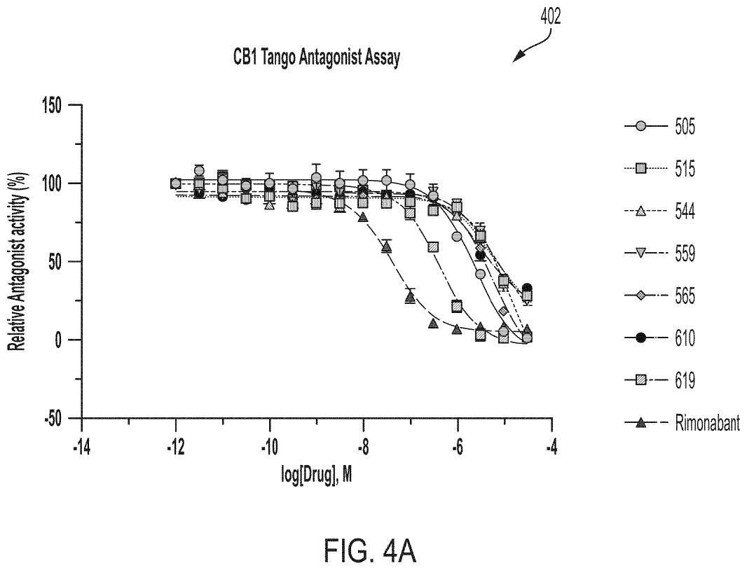

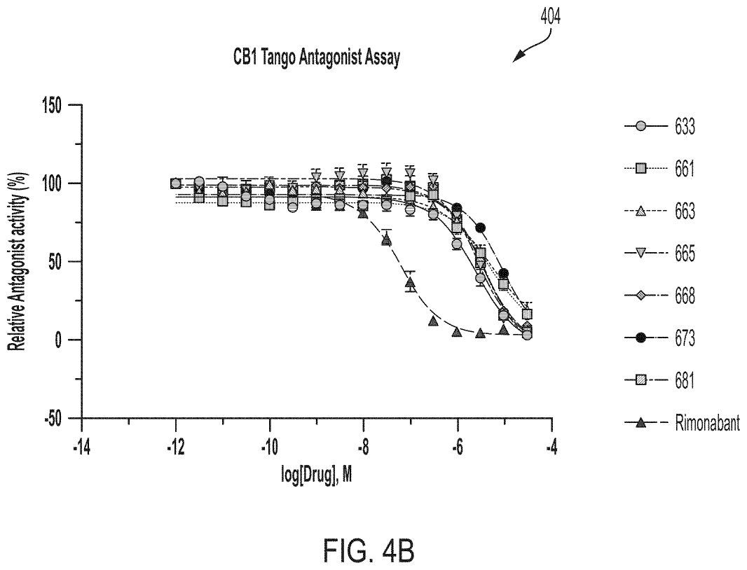

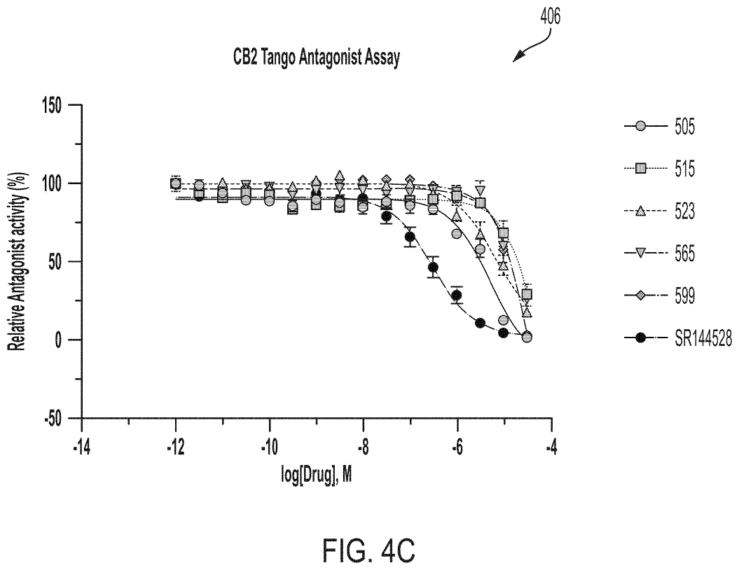

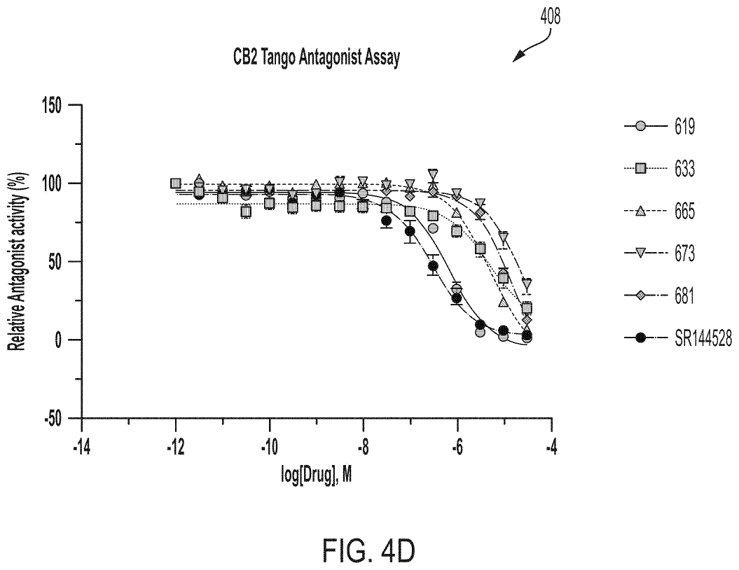

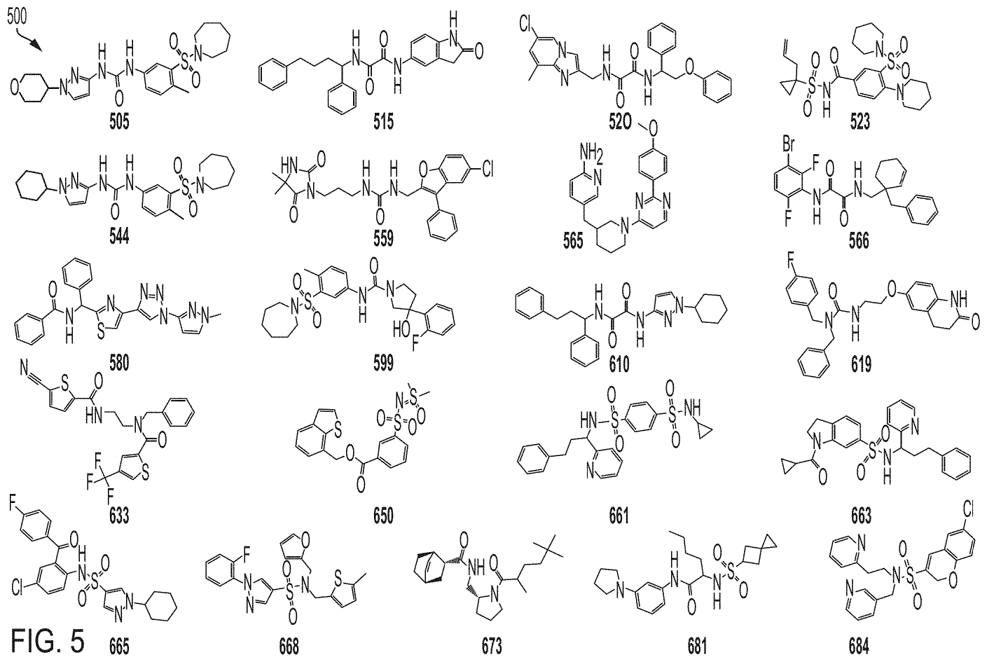

The approach herein may include identification and characterization of new CB ligands from V-SYNTHES screening. Initial functional characterization of 60 novel candidate ligands predicted by V-SYNTHES identified 21 compounds with antagonist activity (>40% inhibition at 10 μM concentration) at human CB₁, CB₂ or both in the β-arrestin recruitment Tango assay. Only one compound, 673, showed weak CB₂ agonism at 10 μM, though behaved as antagonist at lower concentrations. The initial hits were then further tested for their antagonist potency in full 16-point dose-response assays at CB₁ and CB₂, in the presence of a fixed concentration of the dual CB₁/CB₂ CP55,940 agonist that submaximally activates the receptors (see FIGS. 4A-D). Among the 60 compounds predicted by V-SYNTHES, the Tango assays identified 21 hits with functional Kᵢ values better than 10 μM, including 21 antagonists for CB₁ and 20 antagonists for CB₂ (see Table 1), with their chemical structures 500 presented in FIG. 5. This constitutes a high 33% hit rate for both receptors, on the high end of the range observed in prospective screening for GPCRs. Among identified hit compounds, 14 showed sub-micromolar functional Kᵢ values as antagonists at the CB₁ receptor and 3 compounds at the CB₂ receptor. The same 60 compounds were also tested in radioligand binding assays with human CB₂ and rat CB₁ receptors and [³H]CP-55,940 as the radioligand. Of these, 9 compounds had affinities (Kᵢ) better than 10 μM to CB₁ receptor and 16 compounds with affinities better than 10 μM to CB₂ receptor.

To assess the broad off-target selectivity, the best compounds, 523, 610, and 673, were also tested at 10 μM concentration in GPCRome-Tango assays with the panel of more than 300 receptors. The assay panel shows only a few (3-5) potential actives, while the follow-up dose-response curves reveal only negligible activities at these off-targets.

FIGS. 4A-B depict graphs 402, 404 showing functional characterization of the best V-SYNTHES hits in Tango antagonist assay at CB₁. FIGS. 4C-D depict graphs 406, 408 showing the best hits at CB₂ receptor (c-d) Best hits at CB₂ receptor.

TABLE 1

| | | | CB₁ | | CB₂ | | | | | | |
|---|---|---|---|---|---|---|---|---|---|---|---|
| | | | Antagonist | | Antagonist | | | | | | |
| | | | potency | | potency | | CB₁ affinity | | CB₂ affinity | | |
| | | PDSP | Kᵢ, | 95% | Kᵢ, | 95% | Kᵢ, | 95% | Kᵢ, | 95% | |
| BRI-ID | # | ID | uM | CI | uM | CI | uM | CI | uM | CI | Tanimoto |
| BRI-13505 | 505 | 56707 | 0.28 | 0.22- | 0.54 | 0.43- | 16.4 | 8.6- | 1* | N.D. | 0.38 |
| | | | | 0.36 | | 0.67 | | 31.3 | | | |
| BRI-13515 | 515 | 56731 | 0.94 | 0.76- | 3.81 | 2.89- | 6.1 | 2.9- | 2.85 | 1.9- | 0.39 |
| | | | | 1.16 | | 5.09 | | 13.0 | | 4.1 | |
| BRI-13520 | 520 | 56717 | 1.07 | 0.84- | 5.20 | 3.82- | 11.6 | 3.7- | 12.8 | 4.8- | 0.40 |
| | | | | 1.37 | | 7.22 | | 35.7 | | 34.2 | |
| BRI-13523 | 523 | 56737 | 1.82 | 1.46- | 1.59 | 1.27- | 12.0 | 5.4- | 0.85 | 0.69- | 0.39 |
| | | | | 2.28 | | 1.98 | | 26.7 | | 1.05 | |
| BRI-13544 | [symbol] | 56724 | [symbol] | [symbol] | 7.78 | 4.66- | 5.0- | N.D. | 2.5* | N.D. | 0.34 |
| | | | | [symbol] | | 16.8 | 7.2* | | | | |
| BRI-13559 | 559 | 56715 | 0.98 | 0.80- | 4.25 | 3.15- | N.D.* | N.D. | 12.2 | 2.1- | 0.43 |
| | | | | 1.20 | | 5.90 | | | | 69.6 | |
| BRI-13565 | [symbol] | 56684 | [symbol] | [symbol] | 3.77 | 2.71- | 4.5* | N.D. | 13.6 | 8.4- | 0.37 |
| | | | | [symbol] | | 5.53 | | | | 22.0 | |
| BRI-13566 | 566 | 56708 | 2.05 | 1.63- | 4.04 | 3.02- | 6.9* | N.D. | 1.2 | 0.84- | 0.43 |
| | | | | 2.60 | | 5.48 | | | | 1.57 | |
| BRI-13580 | 580 | 56727 | 5.80 | 4.55- | 6.92 | 5.51- | 1.0- | N.D. | 1.5* | N.D. | 0.36 |
| | | | | 7.55 | | 8.80 | 9.0* | | | | |
| BRI-13599 | 599 | 56723 | 2.33 | 1.82- | 2.44 | 2.06- | 26.5* | N.D. | 10.4 | 7.1- | 0.34 |
| | | | | 3.01 | | 2.89 | | | | 15.1 | |
| BRI-13610 | 610 | 56696 | 0.76 | 0.62- | 4.17 | 3.14- | 0.62 | 0.34- | 0.28 | 0.12- | 0.31 |
| | | | | 0.93 | | 5.62 | | 1.13 | | 0.69 | |
| BRI-13619 | 619 | 56695 | 0.05 | 0.04- | 0.11 | 0.09- | 45* | N.D. | 0.9- | N.D. | 0.42 |
| | | | | 0.06 | | 0.13 | | | 2.5* | | |
| BRI-13633 | 633 | 56726 | 0.23 | 0.19- | 1.53 | 1.18- | 10* | N.D. | 0.7- | N.D. | 0.50 |
| | | | | 0.28 | | 1.98 | | | 0.9* | | |
| BRI-13650 | 650 | 56725 | 3.22 | 2.61- | 12.2 | 7.85- | 45* | N.D. | 0.9- | N.D. | 0.48 |
| | | | | 4.01 | | 20.7 | | | 2.5* | | |
| BRI-13661 | 661 | 56685 | 0.55 | 0.43- | 4.37 | 3.37- | 19* | N.D. | 4.0 | 2.4- | 0.39 |
| | | | | 0.70 | | 5.74 | | | | 6.7 | |
| BRI-13663 | [symbol] | 56687 | [symbol] | [symbol] | 14.5 | 9.89- | 12.5* | N.D. | 14.3 | 6.6- | 0.36 |
| | | | | [symbol] | | 23.0 | | | | 30.9 | |
| BRI-13665 | 665 | 56732 | 0.39 | 0.32- | 0.82 | 0.71- | >7* | N.D. | 6.7 | 4.2- | 0.47 |
| | | | | 0.47 | | 0.95 | | | | 10.6 | |
| BRI-13668 | [symbol] | 56691 | [symbol] | [symbol] | 4.78 | 3.60- | 5.5- | N.D. | 5.2 | 2.5- | 0.40 |
| | | | | [symbol] | | 6.42 | 6.9* | | | 11.0 | |
| BRI-13673 | 673 | 56683 | 0.97 | 0.84- | 3.66 | 2.98- | 4.2 | 2.9- | 2.2 | 1.4- | 0.46 |
| | | | | 1.14 | | 4.51 | | 6.0 | | 3.4 | |
| BRI-13681 | 681 | 56701 | 0.42 | 0.32- | 1.86 | 1.52- | 8.2 | 5.2- | 4.2 | 2.5- | 0.42 |
| | | | | 0.55 | | 2.30 | | 12.7 | | 7.2 | |
| BRI-13684 | 684 | 56689 | 1.16 | 0.93- | 7.28 | 4.50- | 25.5 | 16.6- | 5.3 | 3.5- | 0.48 |
| | | | | 1.43 | | 14.4 | | 39.0 | | 8.1 | |
| SR144528 | N/A | | N.D. | N.D. | 0.052 | 0.041- | | | | | |
| | | | | | | .066 | | | | | |
| Rimonabant | N/A | | 0.006 | 0.005- | N.D. | N.D. | | | | | |
| | | | | .008 | | | | | | | |
| CP55940^{&} | N/A | | 0.017 | | 0.028 | | | | | | |

As mentioned, Table 1 illustrates results of V-SYNTHES hits in functional and binding assays. Sub-micromolar hits are shown in bold, selective by italic. Kᵢ values and 95% Confidence Intervals are calculated from n=4 independent assays with 16 dose-response points. An asterisk marks estimates from 3-point assays. An ampersand marks potency measured in agonist mode. N.D. means “not determined.”

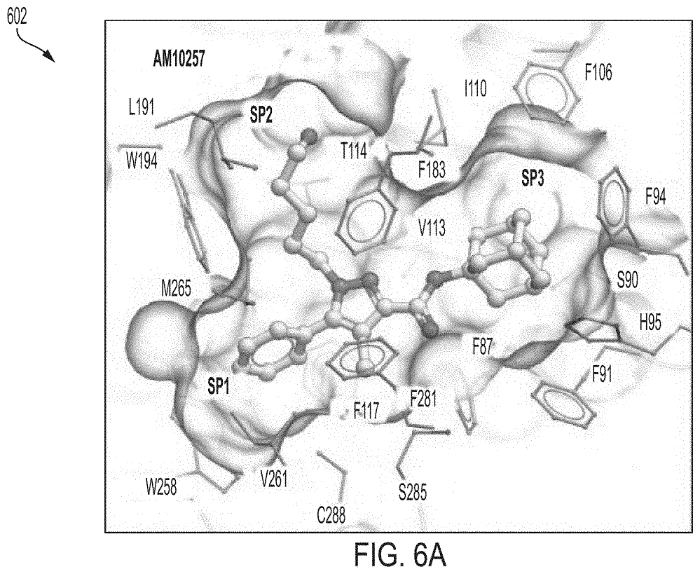

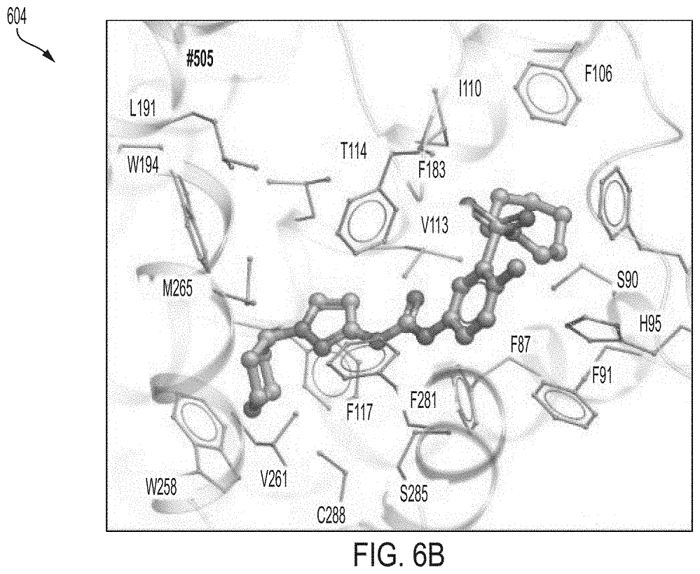

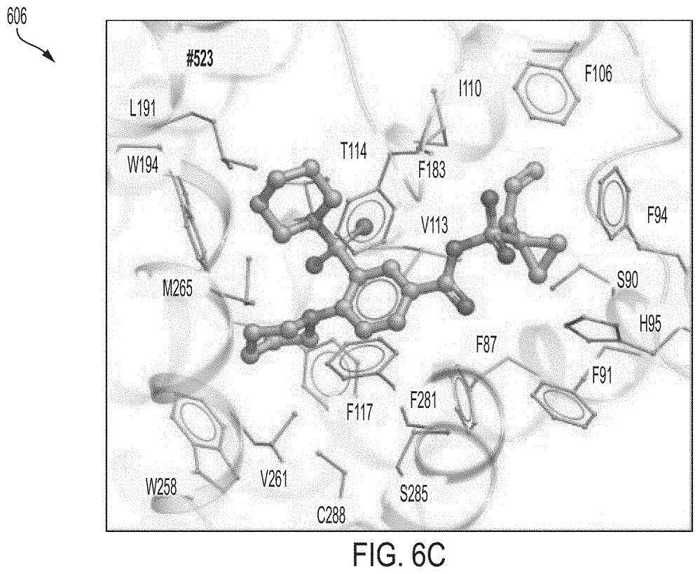

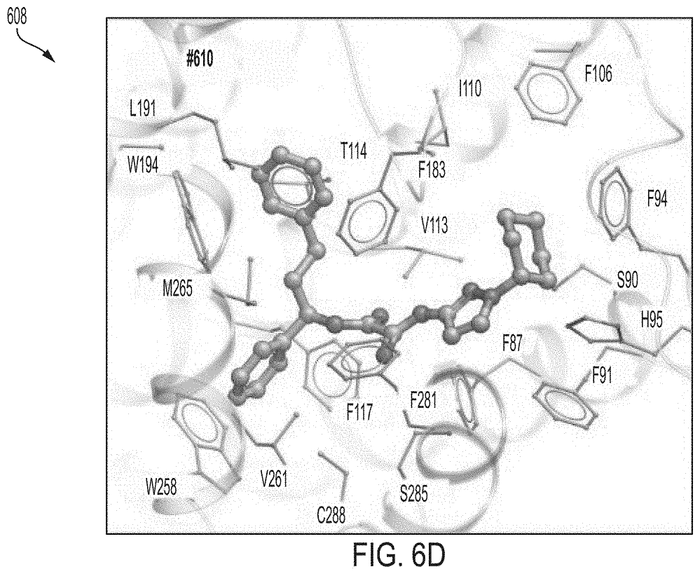

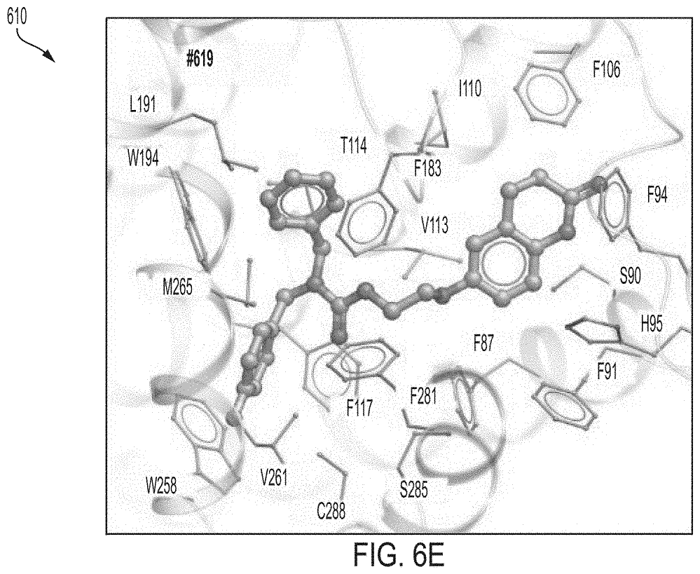

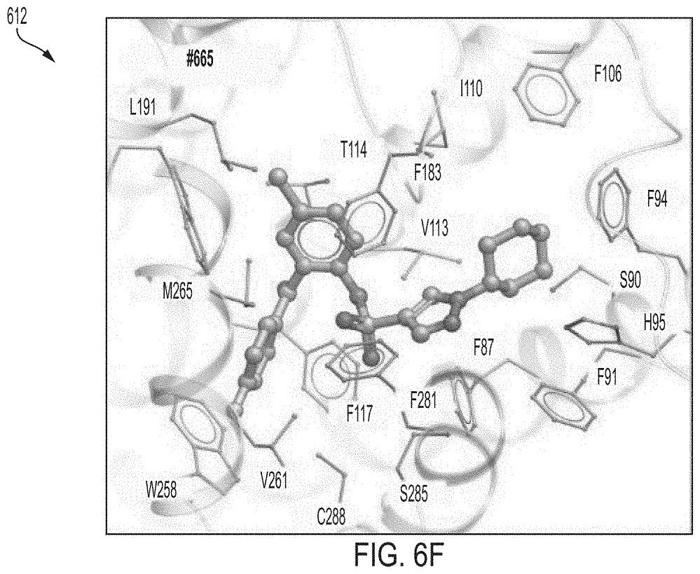

Molecular determinants of the hit compound binding and antagonism are also discussed. With reference to FIG. 5, experimentally identified hit compounds show a broad diversity in their chemical structures 500, representing novel scaffolds with Tanimoto distance >0.3 from known CB₁ and CB₂ ligands found in ChEMBL (pAct >5.0). The best hit compounds are predicted to largely fill the receptor orthosteric pocket, similar to antagonist AM10257 that was co-crystallized with CB₂ receptor (see FIGS. 6A-F). Best hit compounds occupy all three subpockets of the CB₂ binding pocket, where benzene ring (Subpocket 1), 5-hydroxypentyl chain (Subpocket 2), and adamantyl group (Subpocket 3) of AM10257 are bound in the crystal structure of the receptor. Like in AM10257, these interactions suggest antagonistic profiles for our hit compounds, as compared to the recently solved Cryo-EM structure of CB₂ receptor with agonist WIN 55,212-2, which shows that agonist molecules avoid interaction with Subpocket 1 W194, F117, and W258 side chains. Subpocket 1 preferably binds aromatic ring, however, two bit compounds (505 and 523) fill it with a non-aromatic ring and one compound with an aliphatic substituent (681). Interestingly, while most previously known CB₁/CB₂ ligands, including AM10257 and THC analogs have an aliphatic moiety in subpocket SP2, our hits have more bulky cyclic groups in SP2, while compound 505 avoids this pocket altogether. Notably, while lipophilicity of CB receptor pockets represents a challenge for developing high-affinity drug-like ligands, all the V-SYNTHES derived hits have logP<5 and are smaller than 500 DA.

FIG. 6A-F show binding poses for various top CB₂ hits identified by V-SYNTHES. For example, FIG. 6A shows a diagram 602 of a crystal structure of a CB₂ receptor with AM10257. FIG. 6B shows a diagram 604 of a predicted binding pose for hit compound 505. FIG. 6C shows a diagram 606 of a predicted binding pose for hit compound 523. FIG. 6D shows a diagram 608 of a predicted binding pose for hit compound 610. FIG. 6E shows a diagram 610 of a predicted binding pose for hit compound 619. FIG. 6F shows a diagram 612 of a predicted binding pose for hit compound 665. In FIGS. 6A-F, key subpockets of the binding pocket marked as SP1, SP2, and SP3.

In parallel to V-SYNTHES screen, to illustrate performance gains associated with the systems and methods provided herein, a standard ultra-large scale VLS was performed. The standard VLS was performed for a representative 115 million compound subset from Enamine REAL library, using the same receptor model and the same parameters of the docking algorithm. As a result of this standard full-scale screening, 97 predicted hits were selected, synthesized, and tested in the same functional and binding assays as the candidate hits from V-SYNTHES. Out of 97 compounds from standard VLS, 16 compounds shown activity in functional assays, of which 9 compounds were identified as antagonists at CB₁ with functional Kᵢ better or equal to 10 μM, and 5 at CB₂. Of these, 3 compounds had submicromolar antagonist functional Kᵢ at CB₁, and none at CB₂. Binding affinity better than 10 μM was detected for 8 compounds at CB₁ and for 15 at CB₂ (8% and 15% hit rates respectively). Thus, hit rates for the standard VLS did not exceed 15% in any assays, which served as a motivation for the development of the V-SYNTHES approach.

Hits identified using V-SYNTHES have a great potential for further optimization because the combinatorial nature of the vast REAL Space of 11 billion compounds (now 21 billion compounds) ensures thousands of close analogues for structure-activity relationship analysis (SAR). For instance, with returned reference to FIG. 1A, in various embodiments, the method 100 includes performing optimization by structure-activity relationship analysis (SAR) (block 150). A performed structure-activity relationship analysis may include an SAR-by-catalogue search. In an example embodiment, a SAR-by-catalogue search is performed three of the most prominent hits (523, 610 and 673) in REAL Space. A chemical similarity search using ChemSpace fast algorithms selected 920 compounds within a Tanimoto distance of 0.3 from the hits. The hits from the initial V-SYNTHES screening containing the same synthons as the selected hit compounds were also added to the list of similar compounds. On the basis of docking in the same CB2 structural model, 121 of these analogues were selected for synthesis, with 104 of the selected compounds synthesized within 5 weeks. Testing in functional assays detected 60 analogues with a potency that was better than 10 μM and 23 analogues with sub-μM antagonist potency at CB2 (13 for 523 analogues, 7 for 610 and 3 for 673). A series of 523 analogues yielded the most potent antagonists, with at least five compounds (733, 736, 742, 747 and 749) in the low-nM range and more than 50-fold CB2 versus CB1 selectivity in their binding affinity and functional potency. The highest affinity was shown for compound 747 (Ki=0.9 nM). Similar to their parent V-SYNTHES hit 523, the best analogues 33 and 747 also demonstrated high selectivity against the GPCRome—Tango panel of more than 300 receptors. Thus, the V-SYNTHES screen and subsequent SAR-by-catalogue enabled the identification of a CB2-selective lead series with nanomolar activity, good chemical tractability and physico-chemical properties, without requiring custom synthesis.

In addition to the discussion of cannabinoid receptors herein, to assess the broad applicability of the V-SYNTHES approach, further implementations were preformed on the Rho-associated coiled-coil containing protein kinase 1 (ROCK1 or ROCK1 kinase), which is an important and challenging target in cancer drug discovery. A V-SYNTHES screen was performed on 11 billion compounds with minor modifications in the selection procedure. The benchmark comparing the docking of a random compound subset of two-component REAL Space with the docking of selected MEL fragments suggests enrichment EF100≈180 for ROCK1, which is comparable to EF100≈250 obtained for CB screening. 24 fully enumerated compounds were selected and ordered, of which 21 were synthesized and tested for functional potency and binding affinity in human ROCK1 inhibition assays. Potencies of better than 10 μM were found for six compounds (28.5% hit rate), with five of these also showing binding affinities Kd<10 μM in the competitive-binding assay. The best compound, RS-15, achieved potency IC50 =6.3 nM and affinity Kd=7.9 nM.

The discussion herein presents a new modular iterative approach for fast structure-based virtual screening of combinatorial compound libraries, and its application to discovery of novel chemotypes for cannabinoid CB₁ and CB2 receptors among more than 10¹⁰ compounds of Enamine REAL Space. Two assessments of the approach performance were enumerated. In the first, computational performance assessment, V-SYNTHES virtual screen was compared to the standard VLS in the same REAL chemical space. The comparison shows that V-SYNTHES iterations speeds up the identification of 100 hits at a specific binding score threshold about 200-fold for 2-component and 300 fold for 3-component reactions in a test case. The second, more comprehensive assessment compares experimental hit rates for V-SYNTHES with a standard screening of 115M compounds diversity subset from the same Enamine REAL library, using the same docking model and parameters. The best 60 novel and diverse compounds predicted by V-SYNTHES were synthesized using fast high yield parallel reactions and tested in vitro, showing high (˜33%) hit rates for both CB₁ and CB₂ receptors, and identifying 14 submicromolar compounds. This favorably compares to the hit rate (˜9%) obtained by a standard ultra-large VLS screen of ˜115M compounds of the REAL library, which used the same docking model and parameters, but required at least 100 times more computational resources to complete.

The benefits of the V-SYNTHES modular approach, while already obvious with current REAL space libraries, are expected to further increase in the future when the size of virtual libraries becomes even more prohibitive for conventional full screening. In less than a year, the virtual REAL Space grew from ˜11B to more than 15.5B compounds, increasing from 121 to 185 reactions and from 75,000 to 115,000 unique reactants, while maintaining drug-like properties for most of them, synthesis time (5 weeks), and success rate (>80%). The size and diversity of such libraries are expected to grow polynomially with the addition of new optimized reactions and newly available synthons (or building blocks). Thus, the library can grow as fast as N² for the 2-component reactions (where N is the number of synthons), and even faster for 3- and more component reactions. In contrast, the computational cost of the comprehensive V-SYNTHES screen increases only linearly with the number of synthons, and thus can easily accommodate the explosive polynomial growth of REAL libraries to 10¹⁵ and more compounds.

Conceptually, V-SYNTHES takes advantage of a similar paradigm as fragment-based ligand discovery, FBLD, where initial binding of a highly efficient anchor fragment serves as a core for growing the full drug-like compound chemotypes. Classical FBLD, however, requires experimental testing of fragment binding by highly sensitive approaches such as NMR, X-ray or SPR, and thus is limited to smaller libraries (˜1000 compounds) of smaller fragments (<200 DA). The validated fragments are then elaborated by expanding them to fill the binding pocket or connecting several fragments into one molecule, which requires elaborate custom chemistry. In contrast, V-SYNTHES avoids both the experimental testing of weakly binding fragments and custom synthesis of compounds by performing fragment building in very large but well-defined chemical space and yielding lead-like compounds with affinities and potencies reliably measurable by standard biochemical assays. The apparent caveat of skipping experimental validation of initial fragments is a higher reliance on computational docking accuracy. This can, however, be compensated in several ways. First, by using a screen of initial MEL library where most compounds are 250-350 Da, V-SYNTHES also takes advantage of the optimal performance of most docking algorithms, which tend to afford better sampling for smaller, relatively rigid compounds, resulting in the high success of VLS in this range of compound size. Second, V-SYNTHES predicts initial anchor fragments not only for receptor binding, but also for potential utility in further optimization, which is validated by elaborating them to full drug-like molecules. This excludes fragments that are suboptimal in the context of full molecules, or hard to elaborate synthetically from further consideration.

The intrinsic modularity also makes V-SYNTHES approach beneficial not only in initial chemotype discovery but in subsequent optimization of the hits and leads. Because the discovered by V-SYNTHES hits belong to comprehensively covered space of highly modular derivatives, the initial “SAR by catalog” set for the hits can be selected directly in this easily synthesizable space, using fast chemical similarity searches, without requiring elaborate custom synthesis. Notably, the V-SYNTHES screen can be viewed as a “greedy” algorithm, focused on the potentially highest-scoring hits in the libraries of >10 billion compounds. As evaluated by the ultra-large compound screens, a library of 100-200M compounds of similar diversity are likely to contain tens of thousands of reasonable active molecules, so discarding less promising ones for elaboration would be beneficial. Thus, some of the high scoring compounds in a standard VLS may synergistically combine two or more relatively weak synthons (fragments). In contrast, V-SYNTHES give more preference to stronger anchors, selected in the first iteration step. Such compounds with a well-defined strong anchor are likely to have more predictable SAR and be easier to optimize, which may be an additional benefit of V-SYNTHES approach.

Further embodiments of the V-SYNTHES algorithms may include more detailed analysis of several parameters. One such parameter is the criteria for selection of the “blocking” atoms that allow discrimination of “productive” vs. “non-productive” intermediates. This selection may depend on the binding pocket structure and would vary from receptor to receptor, requiring visual analysis of the pocket, and may not be as effective for some types of pockets, e.g. relatively open pockets with less defined subpockets. Also, the balance between enrichment and diversity and the results of the final screen, especially for three- and higher-component reactions may depend on the number of compounds selected on each iterative step while making the library more and more focused. These selection parameters should be set in a way that each iteration selects enough fragments covering diverse chemical space on one hand, on the other hand reducing the number of similar compounds in the screening library on each iteration. Moreover, while the 3-component screen used only ˜1000 top-ranked compounds on the first iteration and ˜5000 on the second to yield 0.5 million compounds set for the final screen, the numbers can be scaled up as needed to achieve as comprehensive coverage as needed.

The newly developed iterative V-SYNTHES approach enables rapid structure-based screening of virtual libraries of 10 Billion and more compounds, such as Enamine REAL Space library. Applied to CB₁ and CB₂ receptors, it enables discovery of high-affinity antagonists with a better success rate than a standard full screening of an ultra-large library, while using —100 times less computational resources to do so. The identified hits have functional potencies as high as 50 nM and are suitable for further optimization in the same easily accessible REAL space. This approach makes ultra-large screening suitable for medium-size CPU clusters and is readily scalable to accommodate the rapid growth of size and diversity of combinatorial virtual libraries.

All reactions in the database of reactions and corresponding synthons can be separated into two categories: 2-components and 3-component reactions, based on the number of variable synthons. For each reaction from the reactions database a Markush structure, representing a reaction scaffold with defined attachment points for substituent synthons, was generated in a smile format. Structures of possible synthons for each R-group in each reaction were generated in 2D format with attachment points defined for enumeration. Enumeration of combinatorial libraries was performed using combinatorial chemistry tools. Markush structures for enumeration were derived from reaction SMARTS.

As mentioned, a Minimal Enumeration Library was generated to generate all possible synthon-scaffold combinations in Enamine REAL Space. Each compound in the MEL library comprises a reaction scaffold enumerated with a single synthon, while other attachment points are replaced with the minimal synthons, or “caps.” Minimal chemically feasible synthons for every substituent in each reaction were selected as either methyl or benzyl, later one in case the reaction required an aromatic group. Minimal synthon atoms were labeled as ¹³C isotope to facilitate the analysis of docking poses. In 2-component Minimal Enumeration Library generation, filters on molecular weight and logP were applied to remove MEL compounds with MW>400 and logP >5, which would likely result in fully enumerated compounds that violate Lipinsi's rule of 5. For 3-component reactions, the size filters were set to MW<350 on the first iteration of V-SYNTHES and to MW<425 on the second.

To generate random subsets of the REAL database for internal benchmarking, enumeration of randomly selected synthons from each reaction was performed. To create the 1 million library of 2-component reactions, 1% of synthons (total of 6418 synthons) were randomly selected, which represented each R group in each reaction. For 3-component reactions, 0.47% of synthons (total of 512 synthons) were randomly selected for the 500K library, with no less than 1 synthon per Markush R group. The random libraries were filtered by Lipinski's rules of five.

To select MEL candidates for further enumeration, the score and docking pose of each MEL candidate were analyzed. The fragments were ranked by score and top 1% were kept for further investigation. To detect “productive” vs. “non-productive” compound poses, the algorithm calculates the distances between the radioactively labeled caps of docked MEL candidates and the selected atoms (or dummy atoms) marking the dead-end sub-pocket in the protein binding site. For CB2 receptor pocket, three dead-end points were used to define potentially “non-productive”MEL ligands: water molecule from the crystal structure and two dummy atoms, one placed between residues F106 and K109, another between residues H95 and L182. MEL compounds for which their cap atoms closer than 4 Å to the “dead-end” points were excluded from further consideration. Furthermore, to ensure diversity of the final library, the best MEL candidates were filtered in a way that the final selection did not contain more than 20% of the MEL candidates from the same reaction.

For 2-component reactions, 819 best MEL candidates were selected for further enumeration resulting in 1M library of full compounds. For 3-component reactions, two rounds of enumerations were required to arrive at full molecules. In the first round, 1043 best MEL candidates were used to produce 500K molecules with two real synthons and one minimal cap. After docking and analysis of these ligands, 4739 best molecules were selected for the final enumeration step resulting in 500K fully enumerated molecules.

Both V-SYNTHES and standard VLS employed a structural model based on CB2R crystal structure with an antagonist AM10257 at 2.8 Å resolution (PDB ID 5ZTY). The structure was converted from PDB coordinates to the internal coordinates object by restoring missing heavy atoms and hydrogens, locally minimizing polar hydrogens, and optimizing His, Asn, and Gln side chains protonation state and rotamers. In the final step of selection, the disclosure also used and ligand-optimized structural models for redocking of top 1% hits. These refined models were generated in a ligand-guided receptor optimization procedure (LiBERO), which refined the sidechains and water molecules within 8 Å radius from the orthosteric binding pocket. Two binding modes for CB2 receptor binding pocket were prepared: one guided by 20 known antagonists and another by 20 agonists, selected from ChEMBL high-affinity ligands for CB2 (CHEMBL253, pK>8). These compounds, along with 200 decoy molecules selected from CB2 receptor decoy database (GDD) were docked into the refined conformers. The conformers yielding the best AUC (Area Under the Curve) ROC (Receiver Operating Characteristics) curves were selected as the best LiBERO models. The two LiBERO models, along with the crystal structure model, were combined into one 4D model as described previously. The 4D model was used for screening in both V-SYNTHES iterative algorithm and standard VLS. Unlike V-SYNTHES, standard VLS used a preassembled library of 115 Million of REAL compounds, including 100M of lead-like subset of REAL and a diversity REAL subset of 15M drug-like compounds.

Docking simulations in both V-SYNTHES and standard VLS were performed using ICM-Pro molecular modeling software (Molsoft LLC). Docking involves an exhaustive sampling of the molecule conformational space in the rectangular box that comprised the CB2 orthosteric binding pocket and was done with the thoroughness parameter set to 2. Docking uses biased probability Monte Carlo (BPMC) optimization of the compound's internal coordinates in the pre-calculated grid energy potentials of the receptor. The 4D model of the receptor pocket described above was used to sample 3 slightly different receptor conformations in a single docking run as implemented in ICM-Pro (Molsoft LLC). Before the final selection of hits for experimental testing the top 30K compounds from the screen were re-docked into the model with higher thoroughness (5) to assure their comprehensive sampling.

To evaluate the efficiency of V-SYNTHES approach and compare it with standard VLS, the discussion introduces an “enrichment factor” that provides a quantitative measurement of how the final library of the method is enriched in hits as compared to a library of the same size generated as a random subset of the Enamine REAL space. For 2-component reactions (500 M compounds), the discussion compares random and enriched libraries of 1M compounds. For 3-component reactions (total 10B compounds), the discussion compared random and enriched libraries of 0.5M compounds. The enrichment is calculated for hits with docking scores equal to or better than a certain threshold X and is defined as the following ratio:

$$Enrichment factor ( X ) = \frac{N of hits with scores < X in SYNTHES}{N of hits with scores < X in standard VLS}$$

The Tango arrestin recruitment assays were performed as previously described. Briefly, HTLA cells were transiently transfected with human CB₁ or CB₂ Tango DNA construct overnight in DMEM supplemented with 10% FBS, 100 μg/ml streptomycin and 100 U/ml penicillin. The transfected cells were then plated into Poly-L-Lysine coated 384-well white clear bottom cell culture plates in DMEM containing 1% dialyzed FBS at a density of 10,000-15,000 cells/well. After 6 hours incubation, the plates were added with drug solutions prepared in DMEM containing 1% dialyzed FBS for overnight incubation. Specially for the antagonist assay, 100 nM of CP55940 was added after 30 minutes of incubation of the drugs. On the day of assay, medium and drug solutions were removed and 20 μL/well of BrightGlo reagent (Promega) was added. The plates were further incubated for 20 min at room temperature and counted using a Wallac TriLux Microbeta counter (PerkinElmer). Results were analyzed using GraphPad Prism 8.

Screening of the compounds in the PRESTO-Tango GPCRome was performed as previously described with modifications. First, HTLA cells were plated in poly-L-lysine coated 384-well white plates in DMEM containing 1% dialyzed FBS for 6 hours. Next, the cells were transfected with 20 ng/well PRESTO-Tango receptor DNAs overnight. Then, the cells were added with 10 μM drugs without changing the medium and incubated for another 24 hours. The remaining steps of the PRESTO-Tango protocol were followed. The results were plotted as fold of basal against individual receptors in the GraphPad 8.0 software. For the receptors that had >3-fold of basal signaling activity, assays were repeated as a full dose-response assay and the results were plotted as a percentage of reference compounds.

The affinities (IQ of the new compounds for rat CB₁ receptor as well as for human CB₂ receptors were obtained by using membrane preparations from rat brain or HEK293 cells expressing hCB₂ receptors, respectively, and [³H]CP-55,940 as the radioligand, as previously described. Results from the competition assays were analyzed using nonlinear regression to determine the IC₅₀ values for the ligand; Kᵢ values were calculated from the I₅₀ (Prism by GraphPad Software, Inc.). Each experiment was performed in triplicate and Kᵢ values determined from three independent experiments and are expressed as the mean of the three values.

Exemplary embodiments of the methods, apparatus, and systems have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

References herein to a computer readable medium, a memory, database, and/or library may include one or more of random access memory (“RAM”), static memory, cache, flash memory and any other suitable type of storage device or computer readable storage medium, which is used for storing instructions to be executed by the processor. The storage device or the computer readable storage medium may be a read only memory (“ROM”), flash memory, and/or memory card, that may be coupled to a bus or other communication mechanism. The storage device may be a mass storage device, such as a magnetic disk, optical disk, and/or flash disk that may be directly or indirectly, temporarily or semi-permanently coupled to the bus or other communication mechanism and used be electrically coupled to some or all of the other components within a computing system including a memory, a user interface and/or a communication interface via a bus.

The term “computer-readable medium” is used to define any medium that can store and provide instructions and other data to a processor, particularly where the instructions are to be executed by a processor and/or other peripheral of the processing system. Such medium can include non-volatile storage, volatile storage and transmission media. Non-volatile storage may be embodied on media such as optical or magnetic disks. Storage may be provided locally and in physical proximity to a processor or remotely, typically by use of network connection. Non-volatile storage may be removable from computing system, as in storage or memory cards or sticks that can be easily connected or disconnected from a computer using a standard interface.

What is claimed is:

1. A method for efficiently screening of large libraries of compounds to identify the best compounds that dock to receptors, for potential use in drug discovery for diseases, the method comprising:
(1) generating a list of proxy compounds comprising reaction scaffolds and enumerated with corresponding synthons only in a first R position while a second R position is capped with a minimal synthon cap to become a capped R position;
(2) docking the proxy compounds to the target receptor structure by docking of a flexible ligand to predict binding scores and ligand-receptor interaction information and to select a first set of best-scoring proxy compounds;
(3) iteratively enumerating the first set of best-scoring proxy compounds so that at least one capped R position is replaced with a full range of corresponding synthons to produce fully enumerated compounds; and
(4) performing docking for the fully enumerated compounds in at least two R positions to select a first set of best docking compounds.

2. The method of claim 1, wherein the minimal synthon cap is methyl or phenyl.

3. The method of claim 1, wherein the first R position is only R1 or only R2 for two-component compounds.

4. The method in claim 1, wherein the large libraries of compounds include Enamine REadily AvailabLe for synthesis (REAL) compound libraries, REAL Space compound libraries, or any other libraries that can be defined as a limited set of Markush scaffolds with two or more R-groups (synthons).

5. The method of claim 1, wherein the second R position is capped with a minimal synthon cap because the reaction scaffolds are often highly polar or charged.

6. The method of claim 1, further comprising filtering or screening the first best set of proxy compounds for diversity.

7. The method of claim 5, wherein the filtering or screening includes an additional compound diversity rule that a single reaction cannot contribute more than 20% of the selection.

8. The method in claim 1, wherein docking the compounds to the target receptor structure further includes selecting of compounds with higher chances for successful enumeration, as defined by distances to specific atoms of a pocket.

9. The method of claim 1, wherein the iteratively enumerating comprises a single iteration for two-component reactions with only two R groups.

10. The method of claim 1, wherein the iteratively enumerating comprises a plurality of iterations for three-component reactions with three R groups.

11. The method of claim 1, wherein the iteratively enumerating comprises repeatedly enumerating a plurality of iterations when the compounds are 4- and 5-component compounds until the compounds are fully enumerated with library synthons.

12. The method of claim 1, wherein the performing the docking for the fully enumerated compounds further includes filtering for physical-chemical properties, drug-likeness, novelty, and chemical diversity to select a final set of best docking compounds for synthesis and testing that is a subset of the first set of best docking compounds.

13. The method of claim 1, wherein the receptors are a cannabinoid CB₁ receptor and a cannabinoid CB₂ receptor.

14. The method of claim 1, wherein the receptors have receptor structures represented by 3D coordinates of the receptor atoms.

15. A computer-readable medium storing instructions that when executed by a processor cause the processor to perform a method for using the computer system to efficiently screening of large libraries of compounds to identify the best compounds that dock to receptors, for potential use in drug discovery for diseases, the method comprising:
(1) generating a list of proxy compounds comprising reaction scaffolds and enumerated with corresponding synthons only in a first R position while a second R position is capped with a minimal synthon cap to become a capped R position;
(2) docking the proxy compounds to the target receptor structure by docking of a flexible ligand to predict binding scores and ligand-receptor interaction information and to select a first set of best-scoring proxy compounds;
(3) iteratively enumerating the first set of best-scoring proxy compounds so that at least one capped R position is replaced with a full range of corresponding synthons to produce fully enumerated compounds; and
(4) performing docking for the fully enumerated compounds in at least two R positions to select a first set of best docking compounds.

16. The computer-readable medium of claim 15, wherein the minimal synthon cap is methyl or phenyl.

17. The computer-readable medium of claim 15, wherein the first R position is only R1 or only R2 for two-component compounds.

18. The computer-readable medium of claim 15, further comprising filtering or screening the first best set of proxy compounds for diversity.

19. The computer-readable medium of claim 15, wherein the receptors are a cannabinoid CB i receptor and a cannabinoid CB₂ receptor.

20. A method for efficiently screening of large libraries of compounds to identify the best compounds that dock to at least one of a cannabinoid CB₁ receptor and a cannabinoid CB₂ receptor, the method comprising:
generating a list of proxy compounds comprising reaction scaffolds and enumerated with corresponding synthons in a first R position and a synthon cap in second R position comprising a capped R position;
docking the proxy compounds to at least one of a cannabinoid CB₁ receptor and a cannabinoid CB₂ receptor by docking of a flexible ligand to select a first set of best-scoring proxy compounds;
iteratively enumerating the first set of best-scoring proxy compounds so that at least one capped R position is replaced with a full range of corresponding synthons to produce fully enumerated compounds; and
performing docking of the fully enumerated compounds in at least two R positions to select compounds that dock to at least one of a cannabinoid CB₁ receptor and a cannabinoid CB₂ receptor.
